(19) United States
(12) Patent Application Publication
(10) Pub. No.: US 2017/0056231 A1
(43) Pub. Date: Mar. 2, 2017

(54) PAD AND SUPPORTER

(71) Applicant: OKAMOTO CORPORATION, Kitakatsuragi-gun (JP)

(72) Inventors: Susumu HARA, Kitakatsuragi-gun (JP); Naomi HAMAZAKI, Kitakatsuragi-gun (JP); Atsushi TOMOHISA, Kitakatsuragi-gun (JP); Tsuneo KAWAGOE, Kitakatsuragi-gun (JP)

(21) Appl. No.: 15/251,719

(22) Filed: Aug. 30, 2016

(30) Foreign Application Priority Data
Aug. 31, 2015 (JP) 2015-170217

(51) Int. Cl. A61F 5/01

(52) U.S. Cl. CPC A61F 5/0111

(57) ABSTRACT

The present invention provides a pad capable of effectively curbing fall of a plantar arch. A pad in accordance with the present embodiment, for supporting the plantar arch of a sole of a foot, is provided in a supporter, made of a stretch cloth, including a plantar part and an instep part, which is connected to the plantar part. The pad is located in the plantar part and has an apex, which is provided at a position corresponding to an area in the sole of the foot which area corresponds to the navicular bone of the foot.

This non-provisional application claims priority under 35 U.S.C. §119 on Patent Application No. 2015-170217 filed in Japan on Aug. 31, 2015, the entire contents of which are hereby incorporated by reference.

### TECHNICAL FIELD

The present invention relates to (i) a pad adapted for use in prevention and treatment of diseases such as plantar fasciitis and (ii) a supporter including the pad.

### BACKGROUND ART

A foot of a human is made up of a plurality of bones combined in the shape of a bow (arch), so that the bones of the foot bear the weight of the human. In addition, a plantar fascia (also called plantar aponeurosis) shaped like a bowstring is formed along a plantar arch. However, excessive stretch of the plantar fascia due to long hours of exercise such as a walk or a running results in inflammation of the plantar fascia. The plantar fascia suffering from inflammation can lose its arch form and have a fallen arch. This can cause plantar fasciitis (plantar aponeurositis).

In order to prevent fall of the plantar arch, which is a cause of the plantar fasciitis, there have been conventionally proposed various kinds of orthoses each equipped with a pad in a position corresponding to the arch of the sole of the foot (Patent Literatures 1 to 4).

A pad-equipped insole as disclosed in Patent Literature 1 cannot produce effectiveness of the pad for pressing up against the arch when shoes are not worn and when the foot is not in contact with the ground (e.g., during indoor activities of daily living and during sleep).

Patent Literature 2 discloses a supporter that compresses an arch and other area such as an ankle by means of a pad-equipped belt. Such a supporter may cause blocked blood circulation and a pain due to compression of the ankle and other area that is not the arch.

In contrast, a pad-equipped strap disclosed in Patent Literature 3 and pad-equipped socks disclosed in Patent Literature 4 can continuously produce effectiveness of the pad for pressing up against the arch while minimizing compression of areas other than the arch.

### CITATION LIST

### Patent Literatures

### [Patent Literature 1]

PCT International Publication No. WO 1999/033417 (Publication date: Jul. 8, 1999)

### [Patent Literature 2]

Japanese Patent Application Publication Tokukai No. 2009-125326 (Publication date: Jun. 11, 2009)

### [Patent Literature 3]

Japanese Unexamined Patent Application Publication (Translation of PCT Application) No. 10-531715 (Tokuhyohei 10-512461) (Publication date: Dec. 2, 1998)

### [Patent Literature 4]

Japanese Registered Utility Model No. 3101370 (Registration date: Feb. 18, 2004)

### SUMMARY OF INVENTION

### Technical Problem

Here, bones of the foot are combined with complexity. Misalignment of a major bone of the foot can collapse the combination of the foot bones in their entirety. This results in fall of the plantar arch. In order to prevent fall of the plantar arch, it is thus important that a pad supports an appropriate area of the sole of the foot, with consideration of structure and characteristics of the bones of the foot.

However, the shapes of the pads disclosed in Patent Literatures 3 and 4 fail to reflect the structure of the bones of the foot. Thus, the pads disclosed in Patent Literatures 3 and 4 cannot sufficiently curb fall of the plantar arch.

The present invention has been accomplished in view of the above problems with the conventional orthoses, and it is an object of the present invention to provide (i) a pad capable of effectively curbing fall of the plantar arch so that it can contribute to prevention and treatment of diseases such as plantar fasciitis and (ii) a supporter including the pad.

### Solution to Problem

In order to solve the above problems, a pad in accordance with the present invention is a pad for supporting a plantar arch of a sole of a foot, the pad being provided in a supporter including: a main body part, made of a stretch cloth, including a plantar part and an instep part, which is connected to the plantar part, the pad being located in the plantar part, the pad including: an apex, which is provided at a position corresponding to an area in the sole of the foot which area corresponds to the navicular bone of the foot.

### Advantageous Effects of Invention

The present invention yields the effect of enabling the provision of (i) a pad capable of supporting the navicular bone in place to effectively curb fall of the plantar arch so that it can contribute to prevention and treatment of diseases such as plantar fasciitis and (ii) a supporter including the pad.

### BRIEF DESCRIPTION OF DRAWINGS

FIG. 1 is a plan view illustrating a supporter in accordance with Embodiment 1 of the present invention, (a) of FIG. 1 illustrates a back surface side of the supporter, and (b) of FIG. 1 illustrates a front surface side of the supporter.

FIG. 2 is a side view of the supporter illustrated in FIG. 1 in a state of being worn by a foot (right foot), (a) of FIG. 2 illustrates a state of the supporter when viewed from a medial side of the foot, and (b) of FIG. 2 illustrates a state of the supporter when viewed from a lateral side of the foot.

FIG. 3 is a cross-sectional view of the supporter taken along the line A-A′ of (a) of FIG. 1.

(a) of FIG. 4 is a top view of a pad illustrated in FIG. 3, (b) of FIG. 4 is a cross-sectional view of the pad taken along the line B-B′ of (a) of FIG. 4, and (c) of FIG. 4 is a side view of the pad illustrated in (a) of FIG. 4 when viewed from a first inclined surface side.

(a) of FIG. 5 is a side view of a bony framework of the foot (right foot) when viewed from the medial side of the foot, and (b) of FIG. 5 is a bottom view of the bony framework of the foot when viewed from the sole side of the foot.

FIG. 6 is a cross-sectional view illustrating an example method of mounting the pad into the plantar part illustrated in FIG. 1.

FIG. 7 is a plan view illustrating a supporter in accordance with Embodiment 2 of the present invention. (a) of FIG. 7 illustrates a back surface side of the supporter, and (b) of FIG. 7 illustrates a front surface side of the supporter.

FIG. 8 is a side view of the supporter illustrated in FIG. 7 in a state of being worn by a foot (right foot), (a) of FIG. 8 illustrates a state of the supporter when viewed from a medial side of the foot, and (b) of FIG. 8 illustrates a state of the supporter when viewed from a lateral side of the foot.

FIG. 9 is an external view of a supporter in accordance with Embodiment 3 of the present invention.

FIG. 10 is a side view of the supporter illustrated in FIG. 9 in a state of being worn by a foot (right foot), (a) of FIG. 10 illustrates a state of the supporter when viewed from a medial side of the foot, and (b) of FIG. 10 illustrates a state of the supporter when viewed from a lateral side of the foot.

FIG. 11 is an external view of a supporter in accordance with Embodiment 4 of the present invention.

FIG. 12 is a side view of the supporter illustrated in FIG. 11 in a state of being worn by a foot (right foot), (a) of FIG. 12 illustrates a state of the supporter when viewed from a medial side of the foot, and (b) of FIG. 12 illustrates a state of the supporter when viewed from a lateral side of the foot.

### DESCRIPTION OF EMBODIMENTS

### Embodiment 1

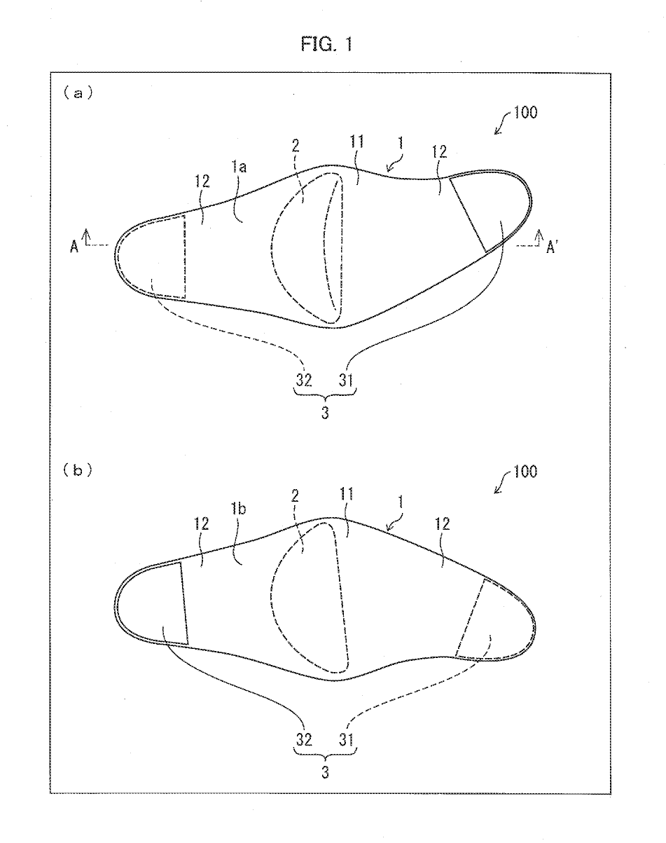

The following description will discuss an embodiment of the present invention with reference to FIGS. 1 to 6. The present embodiment will describe an example supporter that includes a pad in accordance with the present invention.

[Configuration of Supporter 100]

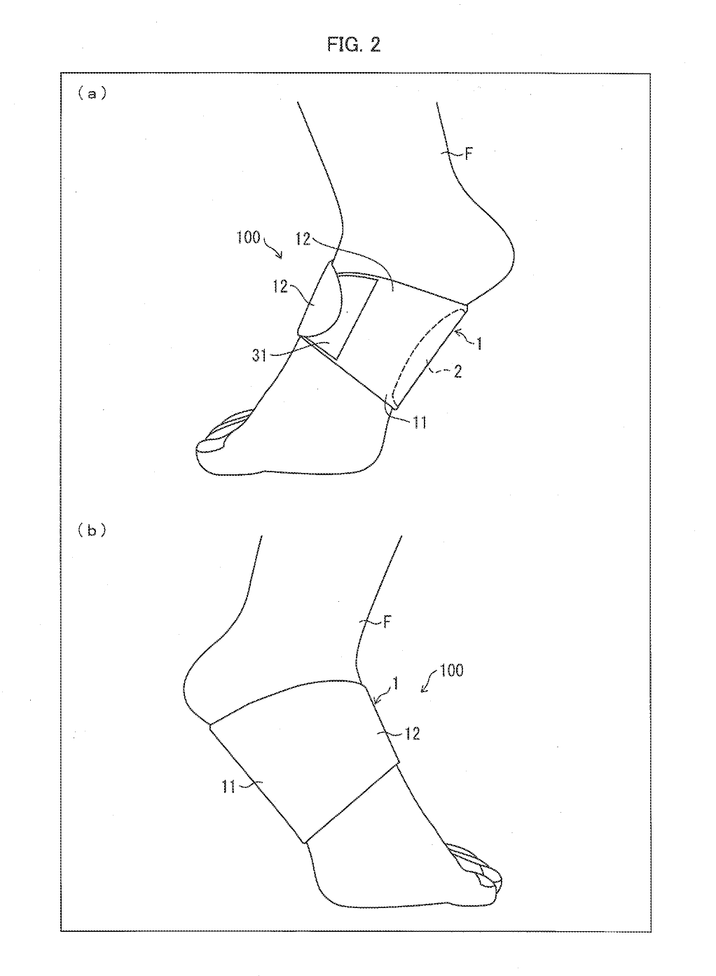

FIG. 1 is a plan view illustrating a supporter 100 in accordance with the present embodiment. (a) of FIG. 1 illustrates a back surface 1a side of the supporter 100, and (b) of FIG. 1 illustrates a front surface 1b side of the supporter 100. FIG. 2 is a side view of the supporter 100 illustrated in FIG. 1 in a state of being worn by a foot (right foot) F. (a) of FIG. 2 illustrates a state of the supporter 100 when viewed from a medial side of the foot F, and (b) of FIG. 2 illustrates a state of the supporter 100 when viewed from a lateral side of the foot F.

The supporter 100 in accordance with the present embodiment has a pad 2 that supports a plantar arch of a sole of the foot F when the supporter 100 in accordance with the present embodiment is wrapped around a circumference of the foot F.

As illustrated in FIGS. 1 and 2, the supporter 100 includes a main body part 1, a pad 2, and a first fastener 3.

(Main Body Part 1)

The main body part 1 is a strap to be wrapped around the circumference of the foot F. The main body part 1 includes: a plantar part 11 to be placed on the sole of the foot F; and an instep part 12 to be placed on an instep of the foot F. The main body part 1 has a shape such that it extends in a direction of a width of the foot F (in a side-to-side direction of FIG. 1). In a case where the main body part 1 is wrapped around the circumference of the foot F, the plantar part 11, which is a central portion of the main body part 1, is placed on the sole of the foot F, while the instep part 12, which is both end portions of the main body part 1, is placed on the instep of the foot F.

The main body part 1 is made of cloth having a high degree of stretch (stretch cloth). The stretch cloth is preferably excellent in air permeability and moisture-absorption property. Preferable examples of the stretch cloth include (i) natural fibers such as cotton, linen, wool, and silk and (ii) chemical fibers such as polyester, nylon, rayon, and acrylic.

The pad 2 is mounted into the plantar part 11. The supporter 100 is wrapped around the circumference of the foot F while the stretchable main body part 1 is stretched out. The supporter 100 supports the plantar arch with the pad 2 being pressed against the sole of the foot F by the main body part 1 so as to support a navicular bone of the foot F.

The main body part 1 is such that an area of the instep part 12 (width of the instep part 12 in the direction of the longitudinal length of the foot F) is smaller than an area of the plantar part 11 (width of the plantar part 11 in the direction of the longitudinal length of the foot F). A decrease in area of the stretch cloth of the instep part 12 decreases a compression force. This makes it possible to reduce a pressure exerted on the instep of the foot F.

(Pad 2)

The pad 2 supports the navicular bone of the foot F in place to effectively curb fall of the plantar arch. The pad 2 has shape restoration property. In other words, the pad 2 alters its shape when a load is applied thereto and restores its shape when the load is removed.

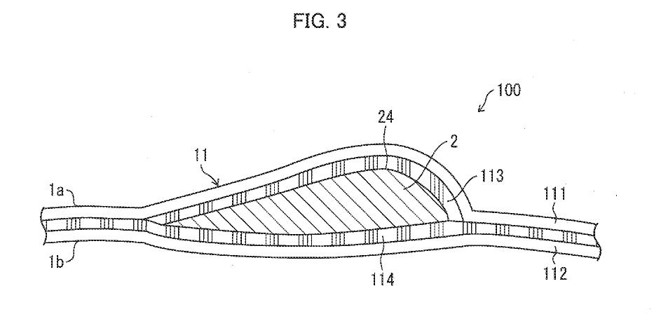

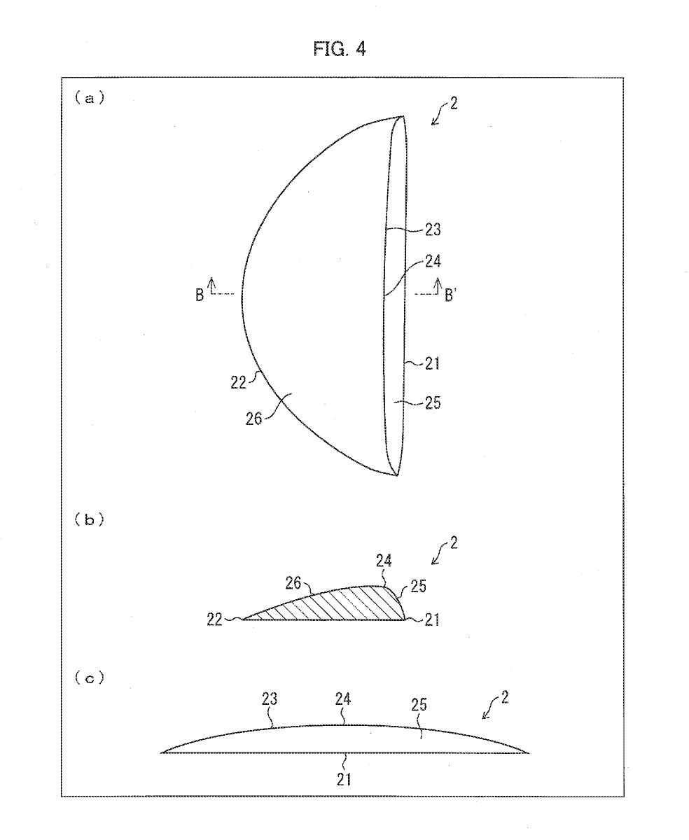

FIG. 3 is a cross-sectional view of the supporter 100 taken along the line A-A′ of (a) of FIG. 1. As illustrated in FIG. 3, the pad 2 is placed in the plantar part 11 with an apex 24, which is the highest part of the pad 2, raised toward the back surface 1a side of the plantar part 11 (toward a side of the plantar part 11 on which side the plantar part 11 contacts the foot F).

Specifically, the pad 2 is provided between a first stretch cloth (stretch cloth) 111, which forms the back surface 1a of the plantar part 11, and a second stretch cloth 112 (stretch cloth), which forms the front surface 1b of the plantar part 11.

A first low-resilience cloth (low-resilience cloth) 113 is interposed between the pad 2 and the first stretch cloth 111. Further, a second low-resilience cloth (low-resilience cloth) 114 is interposed between the pad 2 and the second stretch cloth 112. The first low-resilience cloth 113 and the second low-resilience cloth 114 are each made from a material having resilience lower than that of the pad 2, such as a polyurethane sheet.

The first low-resilience cloth 113, which is interposed between the pad 2 and the first stretch cloth 111, and the second low-resilience cloth 114, which is interposed between the pad 2 and the second stretch cloth 112, can absorb a shock exerted on the sole of the foot F during a walk or other motion. With this arrangement, a burden on the foot F is reduced, and the plantar part 11 conforms to irregular shapes of the sole of the foot F. This achieves a better fit of the supporter 100.

Note, however, that both of the first low-resilience cloth 113 and the second low-resilience cloth 114 are not necessarily provided in the main body part 1. Alternatively, any one of the first low-resilience cloth 113 and the second low-resilience cloth 114 may be provided in the main body part 1.

The pad 2 is made of a material such as wool, polyurethane, latex, polyethylene, ester, polyamide, polyolefin, polyvinyl chloride, or silicone. From the viewpoint of processability, the pad 2 is preferably made of polyurethane and is particularly preferably made of polyurethane foam. A pad 2 using polyurethane foam achieves (i) an improvement in air permeability and (ii) weight reduction.

If the pad 2 decreases its shape restoration property with use, the pad 2 might improperly support the navicular bone of the foot F. In order to achieve a repeated use of the pad 2, the pad 2 is preferably made of a material that is less likely to decrease its shape restoration property with use.

Further, the pad 2 is preferably made of a material not being depressed so much under a high load, while exhibiting appropriate flexibility and appropriate elasticity under a low load. This allows the pad 2 to properly support the navicular bone of the foot F without causing a discomfort and a pain.

Furthermore, the pad 2 is preferably molded by a machining method. This allows for a uniform density of the pad 2 and thus enables stabilization of a pressure to be exerted on the sole of the foot during wearing of the supporter 100 (hereinafter referred to as “wearing pressure”). The pad 2 will be detailed later.

(First Fastener 3)

The first fastener 3 can fasten the main body part 1, which is in a form of a strap, therewith in such a manner that the main body part 1 is openable. In the present embodiment, a hook-and-loop fastener is used as the first fastener 3. The first fastener 3 includes a first hook part 31 and a first loop part 32.

The first hook part 31 is provided on an area of the instep part 12 which area is located in the back surface 1a on one end side of the main body part 1. Meanwhile, the first loop part 32 is provided on another area of the instep part 12 which area is located in the front surface 1b on the other end side of the main body part 1. By detaching the first hook part 31 and the first loop part 32, the main body part 1 is permitted to be openable at the instep part 12.

The first fastener 3 is not limited to a particular type of fastener, provided that the first fastener 3 can fasten the main body part 1 therewith in such a manner that the main body part 1 is openable. For example, the first fastener 3 may be a hook and eye, a snap button, or other fastener. Note, however, that with the use of the hook-and-loop fastener as the first fastener 3, it is possible to control the strength of a force in compressing the circumference of the foot F. That is why the hook-and-loop fastener is preferably used as the first fastener 3.

With the supporter 100 configured as described above, the pad 2 is constantly pressed against the plantar arch by the main body part 1. This enables the plantar arch to be supported by the pad 2 even when the foot F is off the ground. As such, the navicular bone is kept in a proper position while the supporter 100 is worn by the foot F. As a result, it is possible to continuously obtain the effectiveness for curbing fall of the plantar arch.

[Details of Pad 2]

(a) of FIG. 4 is a top view of the pad 2 illustrated in FIG. 3, (b) of FIG. 4 is a cross-sectional view of the pad 2 taken along the line B-B′ of (a) of FIG. 4, and (c) of FIG. 4 is a side view of the pad 2 illustrated in (a) of FIG. 4 when viewed from a first inclined surface 25 side.

As illustrated in (a) of FIG. 4, the pad 2 is substantially semicircular in shape when viewed from above. The pad 2 includes a first edge 21, which is to be placed on the medial side of the sole of the foot F. The first edge 21 extends substantially linearly along a longitudinal length of the foot F. The pad 2 further includes a second edge 22, which is to be placed on the lateral side of the sole of the foot F. The second edge 22 extends so as to form a part of a circumference of a circle, which part curves toward the medial side of the foot F, and so as to be connected to the first edge 21.

Furthermore, the pad 2 includes a ridge line 23 that is raised toward the sole of the foot F. The ridge line 23 extends substantially linearly along a longitudinal length of the foot F. The pad 2 is configured such that the height (thickness) of the pad 2 becomes maximum at a middle section of the ridge line 23. The middle section of the ridge line 23, which middle section indicates the maximum height of the pad 2, corresponds to the apex 24 of the pad 2.

As illustrated in (b) of FIG. 4, the apex 24 is positioned close to a first edge 21 side when viewed in the cross-section B-B′, which has been cut so as to be perpendicular to the longitudinal length of the foot F and to pass through the apex 24. The pad 2 includes a first inclined surface 25, which is steeply inclined toward the first edge 21 (the medial side of the sole of the foot) from the apex 24. The pad 2 further includes a second inclined surface 26, which is moderately inclined toward the second edge 22 (the lateral side of the sole of the foot) from the apex 24.

Note that the first inclined surface 25, which generally connects the apex 24 and the first edge 21, has an inclination (mean inclination) relatively larger than that of the second inclined surface 26. Meanwhile, the second inclined surface 26, which generally connects the apex 24 and the second edge 22, has an inclination (mean inclination) relatively smaller than that of the first inclined surface 25. Alternately, the inclinations of the first inclined surface 25 and the second inclined surface 26 may be individually changed in a stepwise manner (e.g., inclinations in two steps).

As illustrated in (c) of FIG. 4, the pad 2 is configured such that the height (thickness) of the pad 2 becomes maximum at the middle section of the ridge line 23. When the pad 2 is viewed from the first inclined surface 25 side, the pad 2 is shaped such that the ridge line 23 is moderately inclined from the apex 24 in the direction of the longitudinal length of the foot F (in a side-to-side direction of (c) of FIG. 4) so as to be symmetric with respect to the apex 24.

(a) of FIG. 5 is a side view of a bony framework of the foot (right foot) F when viewed from the medial side of the foot F, and (b) of FIG. 5 is a bottom view of the bony framework of the foot F when viewed from the sole side of the foot F.

As illustrated in (a) and (b) of FIG. 5, bones of the foot are combined with complexity. Given the structure of the foot bones combined with complexity, misalignment of the navicular bone leads to collapse of a combination of a medial cuneiform bone, an intermediate cuneiform bone, a lateral cuneiform bone, and a talus, which are connected to the navicular bone. As a result, the plantar arch is likely to fall. Therefore, in order to prevent fall of the plantar arch, it is particularly important to support the navicular bone in an optimum position.

Given that fact, the pad 2 has the apex 24, which is provided at a position corresponding to an area in the sole of the foot which area corresponds to the navicular bone of the foot. This allows the apex 24 to support the navicular bone and thus enables the pad 2 to suitably curb misalignment of the navicular bone.

Particularly, it is preferable that the apex 24 support the area in the sole of the foot which area corresponds to a navicular tuberosity of the navicular bone. The navicular tuberosity is the innermost region of the navicular bone beneath the sole of the foot. To the navicular tuberosity, a posterior tibial muscle is attached. A tension of the posterior tibial muscle has a great influence on the maintenance of the plantar arch. Given that fact, the apex 24 is arranged to support the navicular tuberosity, which is the innermost region of the navicular bone beneath the sole of the foot. This allows the pad 2 to more suitably curb misalignment of the navicular bone. Accordingly, the pad 2 can more effectively curb fall of the plantar arch.

Note that the first edge 21 of the pad 2 preferably extends substantially linearly along the longitudinal length of the foot F. This makes it possible to curb deformation of a portion of the pad 2 on the first edge 21 side in the direction of the width of the foot F, when the pad 2 is pressed against the sole of the foot F. This allows the pad 2 to suitably support the navicular bone while curbing displacement of the apex 24 due to the deformation of the pad 2 when pressed against the sole of the foot F.

Further, it is preferable that the pad 2 be so shaped as to support not only the area in the sole of the foot which area corresponds to the navicular bone, but also an area in the sole of the foot which area corresponds to a medial cuneiform bone, an intermediate cuneiform bone, a lateral cuneiform bone, and a talus. With this configuration, the pad 2 is pressed against the entire arch of the sole of the foot, thus enabling reliably supporting the plantar arch.

Still further, the pad 2 is preferably configured such that the first edge 21 has a length of not less than 41 mm and not more than 110 mm. The length of a plantar arch is smaller by approximately 38% than the length of a foot. Given that fact, assume that a child's foot length is 110 mm, while an adult's (man's) foot length is 290 mm. In that case, the pad 2 is produced in such a manner that the first edge 21 has a length of not less than 41 mm and not more than 110 mm. The pad 2 thus produced can be accommodated to varying foot lengths from the child's foot length to the adult's foot length.

[Method of Mounting Pad 2]

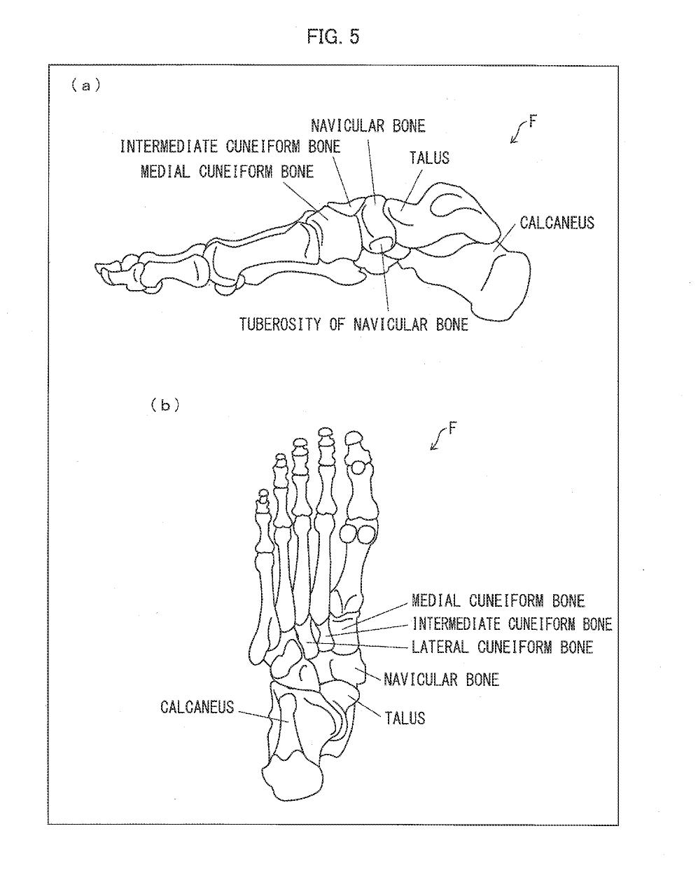

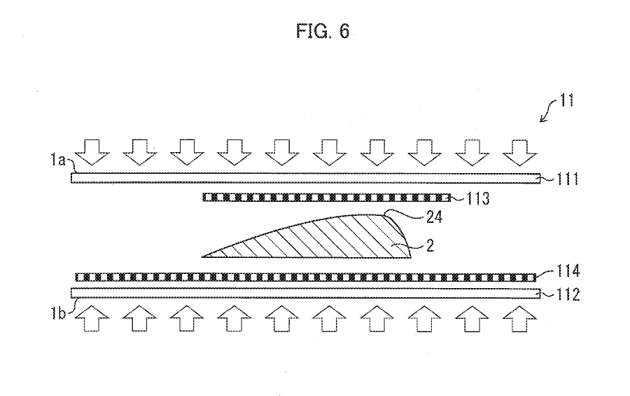

FIG. 6 is a cross-sectional view illustrating an example method of mounting the pad 2 into the plantar part 11. According to the supporter 100, the pad 2 is mounted into the plantar part 11 by molding (thermobonding). Specifically, as illustrated in FIG. 6, the second stretch cloth 112, the second low-resilience cloth 114, the pad 2, the first low-resilience cloth 113, the first stretch cloth 111 are stacked in this order when viewed from the front surface 1b side of the supporter 100. Subsequently, the materials thus stacked are subjected to hot pressing from above and below. As a result, the pad 2 is mounted into the plantar part 11.

As described above, the pad 2 is mounted into the plantar part 11 by molding. This method achieves a higher adhesive strength than the conventional method using an adhesive agent. Conventionally, it is difficult to wash a supporter in which an adhesive agent or the like is used to mount the pad 2 into the plantar part 11, because such a supporter can suffer a significant decrease in adhesive strength after washed. In contrast, the supporter 100, in which the pad 2 is mounted into the plantar part 11 by molding, is less likely to suffer a decrease in adhesive strength after washed. Therefore, the supporter 100 can withstand repeated washings and can be thus kept clean.

(Effect Yielded by Supporter 100)

The supporter 100 in accordance with the present embodiment includes: the main body part 1, made of a stretch cloth, including the plantar part 11 and the instep part 12, which is connected to the plantar part 11; and the pad 2 for supporting a plantar arch. The pad 2 is located in the plantar part 11 and has the apex 24, which is provided at a position corresponding to an area in the sole of the foot F which area corresponds to the navicular bone of the foot F.

According to the supporter 100, the pad 2 has the apex 24, which is provided at a position corresponding to an area in the sole of the foot F which area corresponding to the navicular bone of the foot F. With this arrangement, the apex 24 supports the navicular bone. This allows the pad 2 to suitably curb downward misalignment of the navicular bone.

According to the present embodiment, it is thus possible to achieve the supporter 100 that effectively curbs fall of a plantar arch so that it can contribute to prevention and treatment of diseases such as plantar fasciitis.

### Embodiment 2

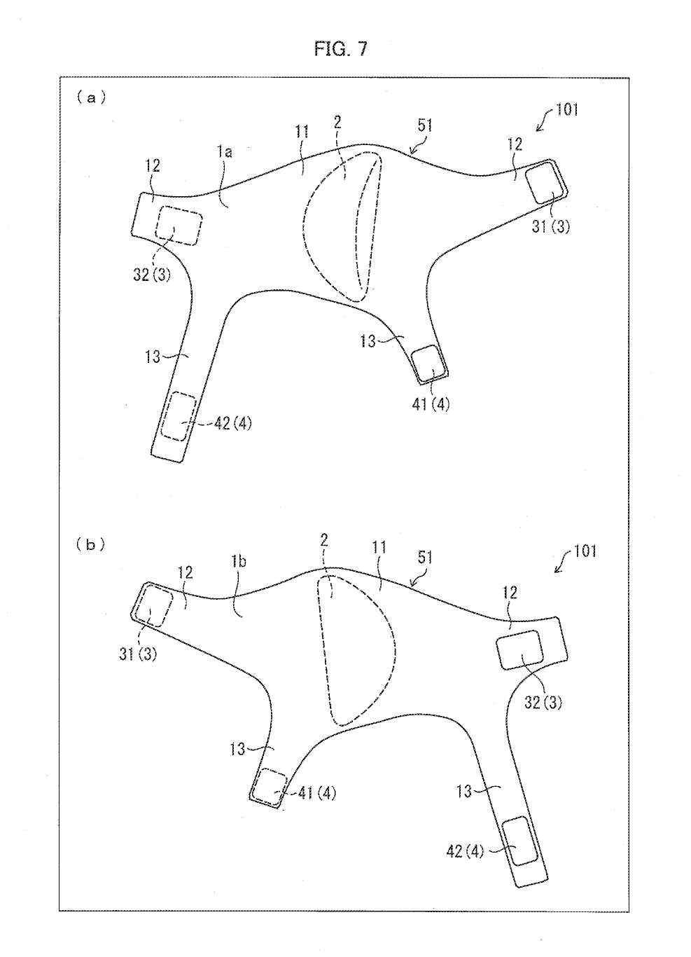

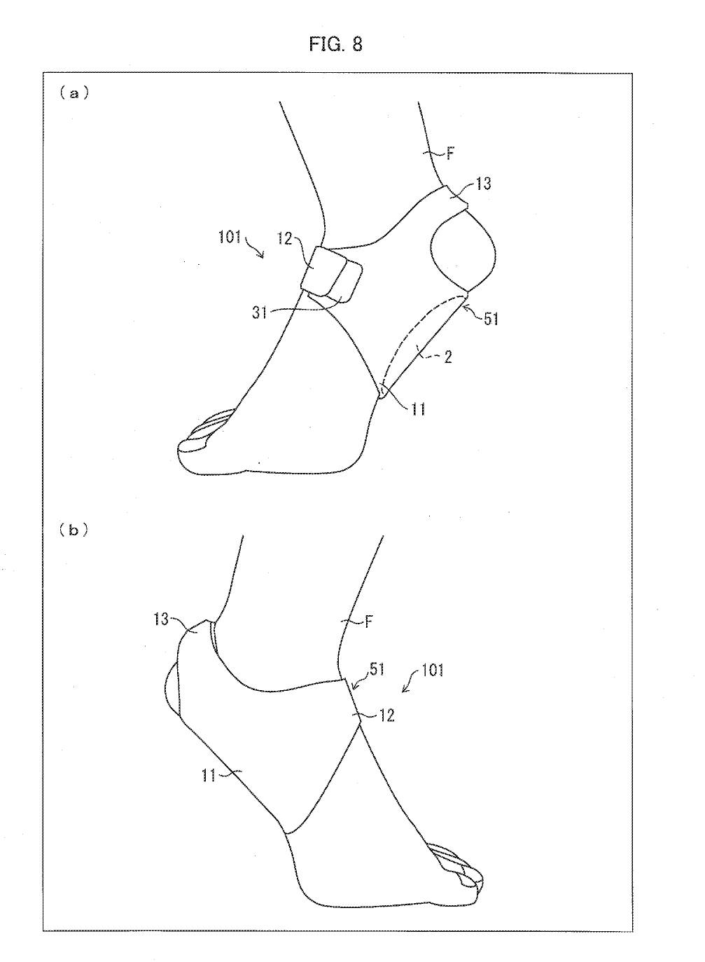

The following description will discuss another embodiment of the present invention with reference to FIGS. 7 and 8. The present embodiment will describe another example supporter that includes a pad in accordance with the present invention.

For convenience of explanation, members having the same functions as those described in the aforementioned embodiment are given the same reference numerals, and are not described here.

[Configuration of Supporter 101]

FIG. 7 is a plan view illustrating a supporter 101 in accordance with the present embodiment. (a) of FIG. 7 illustrates a back surface 1a side of the supporter 101, and (b) of FIG. 7 illustrates a front surface 1b side of the supporter 101. FIG. 8 is a side view of the supporter 101 illustrated in FIG. 7 in a state of being worn by a foot (right foot) F. (a) of FIG. 8 illustrates a state of the supporter 101 when viewed from a medial side of the foot F, and (b) of FIG. 8 illustrates a state of the supporter 101 when viewed from a lateral side of the foot F.

As illustrated in FIGS. 7 and 8, the supporter 101 includes a main body part 51, a pad 2, a first fastener 3, and a second fastener 4.

(Main Body Part 51)

The main body part 51 is a strap to be wrapped around the circumference of the foot F. The main body part 51 includes: a plantar part 11 to be placed on the sole of the foot F; an instep part 12 to be placed on the instep of the foot F; and an ankle securing part 13 to be wrapped around an ankle. The ankle securing part 13 is provided with the second fastener 4. The ankle securing part 13 is permitted to be openable by the second fastener 4. The ankle securing part 13 is wrapped around the circumference of the ankle when the supporter 101 is worn by the foot F.

(Second Fastener 4)

The second fastener 4 can fasten the ankle securing part 13 therewith in such a manner that the ankle securing part 13 is openable. In the present embodiment, a hook-and-loop fastener is used as the second fastener 4. The second fastener 4 includes a second hook part 41 and a second loop part 42.

The second hook part 41 is provided on an end portion of the ankle securing part 13 on the back surface 1a side of the main body part 51. Meanwhile, the second loop part 42 is provided on another end portion of the ankle securing part 13 on the front surface 1b side of the main body part 51. By detaching the second hook part 41 and the second loop part 42, the ankle securing part 13 is permitted to be openable by the second fastener 4.

(Effect Yielded by Supporter 101)

The supporter 101 in accordance with the present embodiment is configured such that the main body part 51 includes the ankle securing part 13 to be wrapped around the ankle.

According to the supporter 101, the main body part 51 includes the ankle securing part 13 to be wrapped around the ankle. This makes it possible to curb displacement of the main body part 51 when the supporter 101 is worn by the foot.

According to the present embodiment, it is thus possible to achieve the supporter 101 that includes the pad 2 being capable of properly supporting the navicular bone while the pad 2 is fixed in an optimum position.

### Embodiment 3

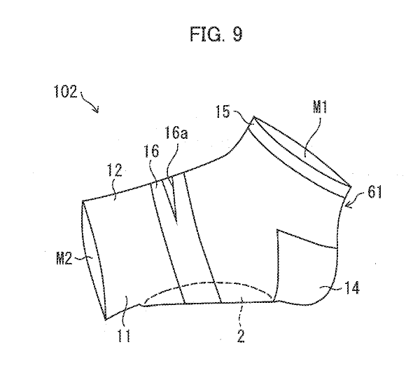

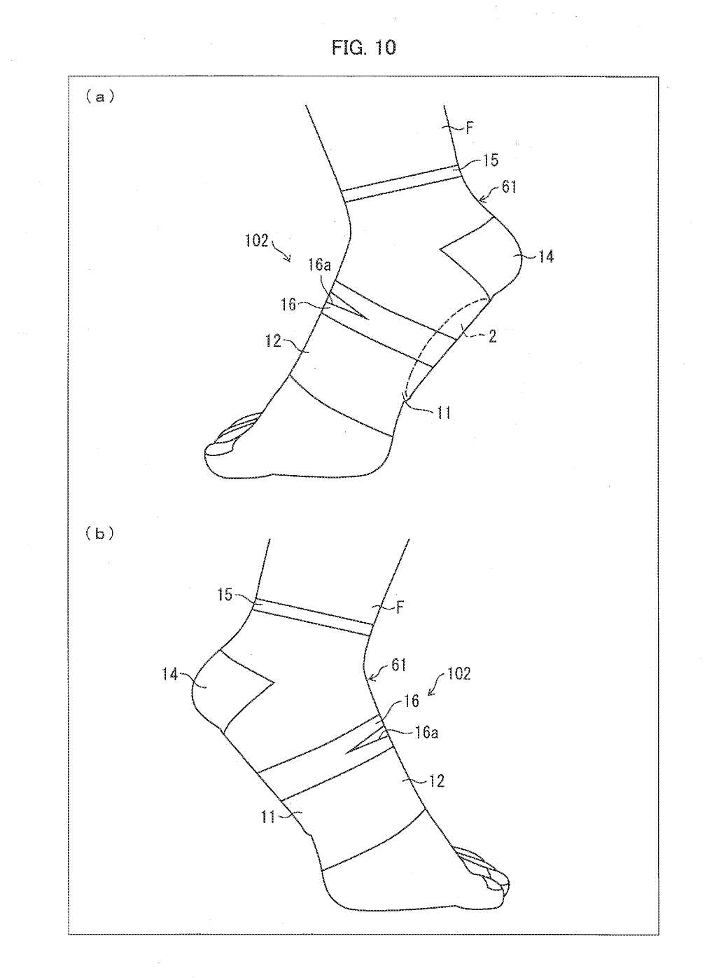

The following description will discuss still another embodiment of the present invention with reference to FIGS. 9 and 10. The present embodiment will describe still another example supporter that includes a pad in accordance with the present invention.

For convenience of explanation, members having the same functions as those described in the aforementioned embodiments are given the same reference numerals, and are not described here.

[Configuration of Supporter 102]

FIG. 9 is an external view of a supporter 102 in accordance with the present embodiment. FIG. 10 is a side view of the supporter 102 illustrated in FIG. 9 in a state of being worn by a foot (right foot) F. (a) of FIG. 10 illustrates a state of the supporter 102 when viewed from a medial side of the foot F, and (b) of FIG. 10 illustrates a state of the supporter 102 when viewed from a lateral side of the foot F.

As illustrated in FIGS. 9 and 10, the supporter 102 includes a main body part 61 and a pad 2.

(Main Body Part 61)

The main body part 61 includes: a plantar part 11 to be placed on the sole of the foot F; an instep part 12 to be placed on the instep of the foot F; a heel part 14 to be placed on the heel of the foot F; an opening part 15 to be placed around the ankle of the foot F; and a ring-shaped compression part 16, which is provided over the plantar part 11 and the instep part 12.

The main body part 61 is made of a tubular stretch cloth having a top opening M1 into which the foot F is inserted and a toe opening M2 from which the toes of the foot F come out. The main body part 61 is knitted in a tubular form by a weft-knitting machine so that the main body part 61 can be controlled in its degree of stretch.

The compression part 16 is provided over the plantar part 11 and the instep part 12 and is made of a ring-shaped cloth extending in the direction of the circumference of the foot F. The compression part 16, which is made of a cloth having stretch lower than those of the plantar part 11 and the instep part 12, pushes the pad 2 toward the navicular bone.

The compression part 16 has a slit 16a provided in an area on the instep part 12 side. With this arrangement, a portion of the compression part 16 on the instep part 12 side is smaller in area than another portion of the compression part 16 on the plantar part 11 side. This allows for reduction of a pressure exerted on the instep of the foot F by the compression part 16. It is thus possible to push the pad 2 toward the navicular bone without blocking blood flows of blood vessels that are dense in the instep of the foot F.

In the present embodiment, the slit 16a is provided in a portion of the compression part 16 on the instep part 12 side to reduce a pressure exerted on the instep of the foot F. Alternatively, for example, the compression part 16 may have different stitch densities so that a wearing pressure exerted on the instep of the foot F is smaller than a wearing pressure exerted on the sole of the foot F.

[Effect Yielded by Supporter 102]

The supporter 102 in accordance with the present embodiment is configured such that the main body part 61 includes the plantar part 11, the instep part 12, the heel part 14, the opening part 15, and the compression part 16. The compression part 16 is made of a ring-shaped cloth, which is provided over the plantar part 11 and the instep part 12, extending in the direction of the circumference of the foot F, so that the compression part 16 pushes the pad 2 toward the navicular bone.

According to the supporter 102, is it possible to wear the supporter 102 over the foot F without using, for example, hook-and-loop fasteners, i.e., the first fastener 3 and the second fastener 4, and to push the pad 2 toward the navicular bone through the compression part 16 while the supporter 102 is worn by the foot F.

According to the present embodiment, it is thus possible to achieve the supporter 102 capable of suitably curbing misalignment of the navicular bone by pushing the pad 2 toward the navicular bone through the compression part 16.

### Embodiment 4

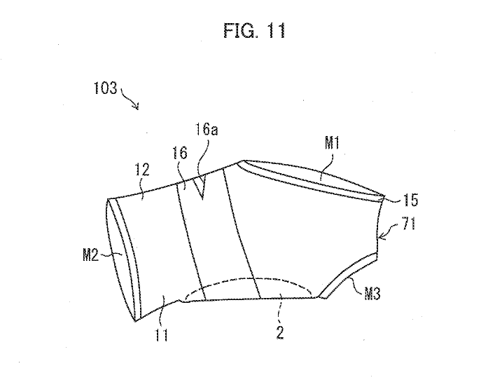

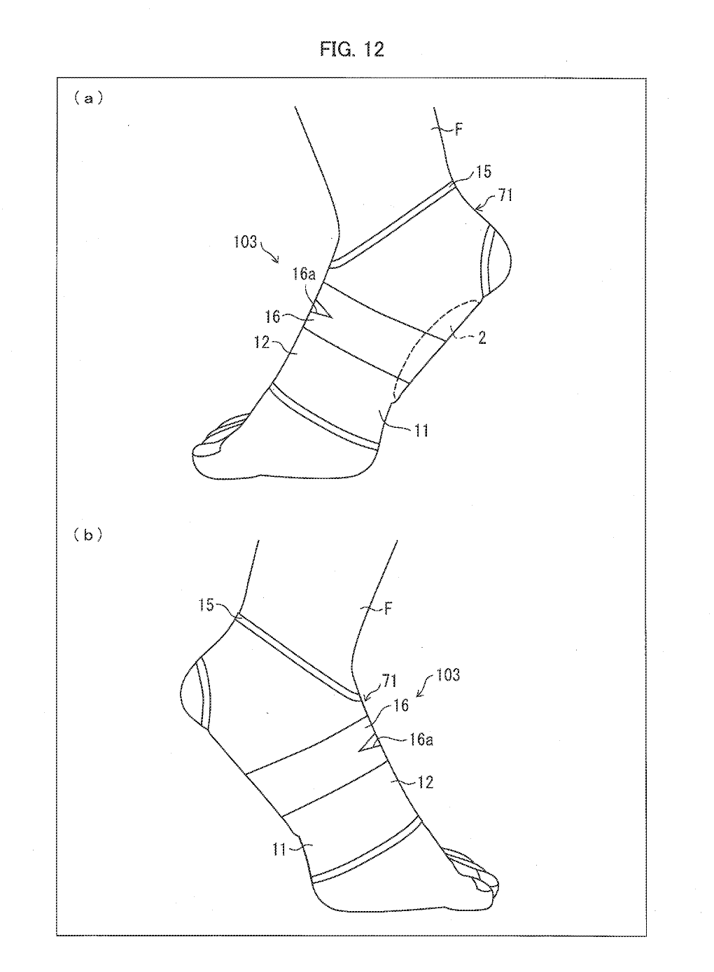

The following description will discuss yet another embodiment of the present invention with reference to FIGS. 11 and 12. The present embodiment will describe yet another example supporter that includes a pad in accordance with the present invention.

For convenience of explanation, members having the same functions as those described in the aforementioned embodiments are given the same reference numerals, and are not described here.

[Configuration of Supporter 103]

FIG. 11 is an external view of a supporter 103 in accordance with the present embodiment. FIG. 12 is a side view of the supporter 103 illustrated in FIG. 11 in a state of being worn by a foot (right foot) F. (a) of FIG. 12 illustrates a state of the supporter 103 when viewed from a medial side of the foot F, and (b) of FIG. 12 illustrates a state of the supporter 103 when viewed from a lateral side of the foot F.

As illustrated in FIGS. 11 and 12, the supporter 103 includes a main body part 71 and a pad 2.

(Main Body Part 71)

The main body part 71 includes: a plantar part 11 to be placed on the sole of the foot F; an instep part 12 to be placed on the instep of the foot F; an opening part 15 to be placed around the ankle of the foot F; and a ring-shaped compression part 16, which is provided over the plantar part 11 and the instep part 12.

The main body part 71 is made of a tubular stretch cloth having a top opening M1 into which the foot F is inserted, a toe opening M2 from which the toes of the foot F come out, and a heel opening M3 from which the heel of the foot F comes out.

The main body part 71 includes not only the toe opening M2 from which the toes of the foot F come out, but also the heel opening M3 from which the heel of the foot F comes out. With this arrangement, the main body part 71 can effectively prevent the foot F from, for example, getting sweaty during the wearing of the supporter 103, as compared to the main body part 61. Further, the top opening M1 of the main body part 71 is larger than that of the main body part 61. This makes the supporter 103 less visible from outside when a shoe is worn over the supporter 103.

[Effect Yielded by Supporter 103]

The supporter 103 in accordance with the present embodiment is configured such that the main body part 71 includes the plantar part 11, the instep part 12, the opening part 15, and the compression part 16. The compression part 16 is made of a ring-shaped cloth, which is provided over the plantar part 11 and the instep part 12, extending in the direction of the circumference of the foot F, so that the compression part 16 pushes the pad 2 toward the navicular bone.

According to the present embodiment, it is thus possible to achieve the supporter 103 capable of suitably curbing misalignment of the navicular bone by pushing the pad 2 toward the navicular bone through the compression part 16.

The present invention is not limited to the embodiments, but can be altered by a skilled person in the art within the scope of the claims. An embodiment derived from a proper combination of technical means each disclosed in a different embodiment is also encompassed in the technical scope of the present invention.

[Recap]

A pad in accordance with the present invention for supporting a plantar arch of a sole of a foot, the pad being provided in a supporter including: a main body part, made of a stretch cloth, including a plantar part and an instep part, which is connected to the plantar part, the pad being located in the plantar part, the pad including: an apex, which is provided at a position corresponding to an area in the sole of the foot which area corresponds to a navicular bone of the foot.

Given the structure of the foot bones combined with complexity, misalignment of the navicular bone can collapse the combination of the foot bones in their entirety. As a result, the plantar arch is likely to fall. More specifically, since the navicular bone is adjacent to many foot bones, misalignment of the navicular bone leads to misalignment of many foot bones. In order to prevent fall of the plantar arch, it is particularly important to support the navicular bone so that the navicular bone is maintained in an optimum position.

According to the above configuration, the pad has the apex, which is provided at a position corresponding to an area in the sole of the foot which area corresponds to the navicular bone of the foot. This allows the apex to properly support the navicular bone, and thus makes it possible to suitably curb misalignment of the navicular bone.

With the above configuration, it is thus possible to achieve a pad that maintains the navicular bone in an optimum position and effectively curbs fall of the plantar arch, so that the pad can contribute to prevention and treatment of diseases such as plantar fasciitis.

Further, the pad in accordance with the present invention is preferably configured such that the apex corresponds to a navicular tuberosity of the navicular bone.

The navicular tuberosity is the innermost region of the navicular bone beneath the sole of the foot. To the navicular tuberosity, a posterior tibial muscle is attached. A tension of the posterior tibial muscle has a great influence on the maintenance of the plantar arch. In view of that fact, an effective way to prevent fall of the plantar arch is to support an area in the sole of the foot which area corresponds to the navicular bone.

According to the above configuration, the apex corresponds to the navicular tuberosity, which is the innermost region of the navicular bone beneath the sole of the foot. With the above configuration, the apex supports the area in the sole of the foot which area corresponds to the navicular bone. This makes it possible to more suitably curb misalignment of the navicular bone.

With the above configuration, it is thus possible to maintain the navicular bone in an optimum position and to effectively curb fall of the plantar arch.

Still further, the pad in accordance with the present invention is preferably configured such that the pad includes a first edge, which is to be placed close to a medial side of the sole of the foot, the first edge extending substantially linearly along a longitudinal length of the foot.

According to the above configuration, the first edge, which is to be placed at a position on the medial side of the sole of the foot, extends substantially linearly along the longitudinal length of the foot. With the above configuration, less unnecessary deformation can occur on a portion of the pad on the first edge side in the direction of the width of the foot, when the pad is pressed against the sole of the foot by the main body part of the supporter, as compared to the conventional pad having a first edge that extends so as to form a part of a circumference of a circle, which part curves toward the lateral side of the sole of the foot.

With the above configuration, it is thus possible to curb misalignment between the apex of the pad and the navicular bone when the pad is pressed against the sole of the foot.

Yet further, the pad in accordance with the present invention may be configured such that the first edge has a length of not less than 41 mm and not more than 110 mm.

The length of a plantar arch is smaller by approximately 38% than the length of a foot. Given that fact, assume that a child's foot length is 110 mm, while an adult's (man's) foot length is 290 mm. In that case, the pad is produced in such a manner that the first edge has a length of not less than 41 mm and not more than 110 mm. The pad thus produced can be accommodated to varying foot lengths from the child's foot length to the adult's foot length.

Further, the pad in accordance with the present invention is preferably configured such that, when the pad is viewed in a cross section which has been cut so as to be perpendicular to the longitudinal length of the foot and to pass through the apex, the pad includes: a first inclined surface, which is steeply inclined toward the medial side of the sole of the foot from the apex; and a second inclined surface, which is moderately inclined toward a lateral side of the sole of the foot from the apex.

With the above configuration, much less unnecessary deformation can occur on a portion of the pad on the first edge side in the direction of the width of the foot, when the pad is pressed against the sole of the foot by the main body part of the supporter.

With the above configuration, it is thus possible to further curb displacement of the apex due to the deformation of the pad when the pad is pressed against the sole of the foot.

Still further, the pad in accordance with the present invention is preferably configured such that the pad supports not only the area in the sole of the foot which area corresponds to the navicular bone, but also an area in the sole of the foot which area corresponds to a medial cuneiform bone, an intermediate cuneiform bone, a lateral cuneiform bone, and a talus.

According to the above configuration, the pad is pressed against the entire arch of the sole of the foot to maintain the navicular bone in an optimum position. This makes it possible to reliably support the plantar arch.

A supporter in accordance with the present invention includes: a pad in accordance with the present invention; and a main body part, made of a stretch cloth, including a plantar part and an instep part, which is connected to the plantar part.

With the above configuration, it is thus possible to achieve a supporter that effectively curbs fall of the plantar arch so that the supporter can contribute to prevention and treatment of diseases such as plantar fasciitis.

Further, the supporter in accordance with the present invention is preferably configured such that the supporter further includes a fastener, provided on the instep part, permitting the main body part to be openable.

According to the above configuration, the fastener provided on the instep part permits the main body part to be openable. This allows the supporter to be easily worn by the foot and removed from the foot. By using, for example, a hook-and-loop fastener as the fastener, it is possible to control a compression force exerted on the foot by the supporter. This makes it possible to optimize a wearing pressure exerted on the sole of the foot by the pad.

Still further, the supporter in accordance with the present invention is preferably configured such that the main body part further includes an ankle securing part to be wrapped around an ankle.

According to the above configuration, the main body part further includes the ankle securing part to be wrapped around the ankle. This makes it possible to curb displacement of the main body part when the supporter is worn by the foot.

With the above configuration, the pad is thus capable of properly supporting the navicular bone while being fixed in an optimum position.

Yet further, the supporter in accordance with the present invention is preferably configured such that the instep part is smaller in area than the plantar part.

According to the above configuration, the area of the stretch cloth in the instep part is smaller. This decreases the strength of the compression force and thus makes it possible to reduce a pressure exerted on the instep of the foot.

With the above configuration, it is thus possible to push the pad toward the navicular bone without blocking blood flows of blood vessels that are dense in the instep of the foot.

Further, the supporter in accordance with the present invention is preferably configured such that a low-resilience cloth is provided between the stretch cloth of the plantar part and the pad, the low-resilience cloth being lower in resilience than the pad.

With this arrangement, a shock exerted on the sole of the foot during a walk or other motion is absorbed by the low-resilience cloth, and the plantar part conforms to irregular shapes of the sole of the foot. This achieves reduction of a burden on the foot and achieves a better fit of the supporter.

Still further, the supporter in accordance with the present invention is preferably configured such that the main body part further includes a ring-shaped compression part, provided over the plantar part and the instep part, having a greater compression force than the stretch cloth and extending in a direction of a circumference of the foot, the compression part pushing the pad toward the navicular bone.

According to the above configuration, the pad is pushed toward the navicular bone through a compression part. This allows the pad to suitably support the navicular bone.

Yet further, the supporter in accordance with the present invention is preferably configured such that a portion of the compression part on the instep part side is smaller in area than another portion of the compression part on the plantar part side, or a wearing pressure exerted on the instep of the foot is lower than a wearing pressure exerted on the sole of the foot.

According to the above configuration, the portion of the compression part on the instep part side has a smaller area. This decreases the strength of the compression force and thus makes it possible to reduce a pressure exerted on the instep of the foot.

With the above configuration, it is thus possible to push the pad toward the navicular bone without blocking blood flows of blood vessels that are dense in the instep of the foot.

[Supplemental Remarks]

The present invention can be expressed as follows. That is, a supporter in accordance with the present invention is a supporter, made of a stretch cloth, including a plantar part and an instep part, the plantar part including a pad part, the pad part being located in an area of the plantar part which area corresponds to a medial cuneiform bone, an intermediate cuneiform bone, a lateral cuneiform bone, a navicular bone, and a talus, the pad part having a thickest portion that corresponds to the navicular bone.

Further, the supporter in accordance with the present invention is preferably configured such that the pad has a medial border that is substantially linear when the supporter is viewed from above.

Still further, the supporter in accordance with the present invention is preferably configured such that the medial border of the pad has a length of 41 mm to 110 mm.

Yet further, the supporter in accordance with the present invention is preferably configured such that inclines of the pad when viewed three-dimensionally are such that the pad has an apex that is a portion which contacts a big toe-side portion of the navicular bone, a medial border is steeply inclined when the pad is viewed from a side surface of the pad, and a lateral border is in a form of a bow when the pad is viewed from above while being moderately inclined when the pad is viewed from the side surface of the pad.

Further, the supporter in accordance with the present invention is preferably configured such that the stretch cloth is in a planer form, and the instep part is in a form of a strap, the instep part including engagement means that makes the supporter tubular when the engagement means is engaged.

Still further, the supporter in accordance with the present invention is preferably configured such that the supporter further includes an ankle securing belt to be provided around an ankle.

Still further, the supporter in accordance with the present invention is preferably configured such that an area of the plantar part is larger than that of the instep part.

Yet further, the supporter in accordance with the present invention is preferably configured such that a low-resilience cloth is provided, in at least a position corresponding to the pad, between the pad and the stretch cloth, which is to be contacted by a skin, the low-resilience cloth being larger in thickness than the stretch cloth.

Further, the supporter in accordance with the present invention is preferably configured such that the main body part is a tubular object, and a strip-shaped compression region is provided around the main body part so as to pass over the center of the pad, the strip-shaped compression region having a higher wearing pressure value than any other region of the main body part.

Still further, the supporter in accordance with the present invention is preferably configured such that a portion of the strip-shaped compression region on an instep side is smaller in area than another portion of the strip-shaped compression region on a sole side.

### REFERENCE SIGNS LIST

1, 51, 61, 71 Main body part
2 Pad
3 First fastener (fastener)
11 Sole part
12 Instep part
16 Compression part
21 First edge
22 Second edge
24 Apex
25 First inclined surface
26 Second inclined surface
31 First hook part (fastener)
32 First loop part (fastener)
100, 101, 102, 103 Supporter
111 First stretch cloth (stretch cloth)
112 Second stretch cloth (stretch cloth)
113 First low-resilience cloth (low-resilience cloth)
114 Second low-resilience cloth (low-resilience cloth)
F Foot (right foot)

1. A pad for supporting a plantar arch of a sole of a foot, the pad being provided in a supporter including: a main body part, made of a stretch cloth, including a plantar part and an instep part, which is connected to the plantar part, the pad being located in the plantar part, the pad comprising:
an apex, which is provided at a position corresponding to an area in the sole of the foot which area corresponds to a navicular bone of the foot.

2. The pad according to claim 1, wherein the apex corresponds to a navicular tuberosity of the navicular bone.

3. The pad according to claim 1, wherein the pad includes a first edge, which is to be placed close to a medial side of the sole of the foot, the first edge extending substantially linearly along a longitudinal length of the foot.

4. The pad according to claim 3, wherein the first edge extending along the longitudinal length of the foot has a length of not less than 41 mm and not more than 110 mm.

5. The pad according to claim 1, wherein, when the pad is viewed in a cross section which has been cut so as to be perpendicular to the longitudinal length of the foot and to pass through the apex, the pad includes: a first inclined surface, which is steeply inclined toward the medial side of the sole of the foot from the apex; and a second inclined surface, which is moderately inclined toward a lateral side of the sole of the foot from the apex.

6. The pad according to claim 1, wherein the pad supports not only the area in the sole of the foot which area corresponds to the navicular bone, but also an area in the sole of the foot which area corresponds to a medial cuneiform bone, an intermediate cuneiform bone, a lateral cuneiform bone, and a talus.

7. A supporter comprising:
a pad recited in claim 1; and
a main body part, made of a stretch cloth, including a plantar part and an instep part, which is connected to the plantar part.

8. The supporter according to claim 7, further comprising:
a fastener, provided on the instep part, permitting the main body part to be openable.

9. The supporter according to claim 7, further comprising:
an ankle securing part to be wrapped around an ankle.

10. The supporter according to claim 7, wherein the instep part is smaller in area than the plantar part.

11. The supporter according to claim 7, wherein a low-resilience cloth is provided between the stretch cloth of the plantar part and the pad, the low-resilience cloth being lower in resilience than the pad.

12. The supporter according to claim 8, wherein a low-resilience cloth is provided between the stretch cloth of the plantar part and the pad, the low-resilience cloth being lower in resilience than the pad.

13. The supporter according to claim 9, wherein a low-resilience cloth is provided between the stretch cloth of the plantar part and the pad, the low-resilience cloth being lower in resilience than the pad.

14. The supporter according to claim 10, wherein a low-resilience cloth is provided between the stretch cloth of the plantar part and the pad, the low-resilience cloth being lower in resilience than the pad.

15. The supporter according to claim 7, wherein the main body part further includes a ring-shaped compression part, provided over the plantar part and the instep part, having a greater compression force than the stretch cloth and extending in a direction of a circumference of the foot, the compression part pushing the pad toward the navicular bone.

16. The supporter according to claim 15, wherein a portion of the compression part on the instep part side is smaller in area than another portion of the compression part on the plantar part side, or a wearing pressure exerted on the instep of the foot is lower than a wearing pressure exerted on the sole of the foot.
